(19) United States
(12) Patent Application Publication
(10) Pub. No.: US 2014/0075337 A1
(43) Pub. Date: Mar. 13, 2014

(54) Dream Sharing and Visualization Platform

(71) Applicant: Richard Anthony Hunter, Kennesaw, GA (US)

(72) Inventor: Richard Anthony Hunter, Kennesaw, GA (US)

(21) Appl. No.: 14/022,220

(22) Filed: Sep. 9, 2013

Related U.S. Application Data
(60) Provisional application No. 61/699,117, filed on Sep. 10, 2012.

(51) Int. Cl. H04L 29/06; A61B 5/16

(52) U.S. Cl. CPC H04L 65/40; A61B 5/16
USPC 715/753

(57) ABSTRACT

Embodiments of the present disclosure may provide a platform for dream sharing and visualization. First, a description of a dream may be received. The description may comprise a text and/or an image. Then the description may be published to the platform. A user may then pay a fee to have the dream visualized. In various embodiments, the description of the dream may be submitted for a vote by other users of the platform. When the dream is submitted to a vote and receives enough votes, the platform may provide a visual representation of the dream to the user who submitted the description of the dream. The visualization may then be published to the platform.

### RELATED APPLICATION

Under provisions of 35 U.S.C. §119(e), the Applicant claims the benefit of U.S. Provisional Application No. 61/699,117, filed on Sep. 10, 2012, which is incorporated herein by reference.

### TECHNICAL FIELD OF INVENTION

The present disclosure relates to the field of content creation, particularly user created content based on the user's subconscious mind.

### BACKGROUND

Every year an absurd tragedy occurs in our lagging market economies. In the United States, an abysmal 80% of product launches may fail because the traditional market research surveys cannot reliably predict consumer behavior. Organizations, such as media companies, video game developers, advertisers, and marketers spend billions of dollars each year on ineffective market research. The purpose of such market research is to provide data on their respective consumers. Organizations use may this data to provide solutions in the form of services and products that fulfill the demands of the consumers. However, these methods of market research may be ineffective. For example, many traditional methods of market research collect data from consumers' conscious mind. Surveys, questionnaires, focus groups, and interviews may all question and engage the consumer at the conscious level. The conscious mind acts as a filter and mediates consumers' true desires.

An interpretation of the basic conclusions of Rational Emotive Behavioral Therapy may suggest that humans make the vast majority of decisions subconsciously. And in fact, it may be said that markets often fail because consumers, unaware of their subconscious desires, may not be able to articulate their deeper desires. As a result, companies fail to reach these unmet hidden needs. For too long, organizations have been asking questions to the wrong part of the mind. In order to understand consumer behavior, it is imperative to understand the subconscious mind.

The subconscious mind is thought to be expressed in dreams, and is seen as the source of automatic thought. The subconscious is more easily influenced by impulses of emotion than by impulses originating solely in the conscious analytical mind. There is much evidence to support the theory that only emotionalized thoughts have any actionable influence upon the subconscious mind and human behavior. Emotion rules the majority of people. Many believe that humans are not thinkers who feel, we are feelers who think. To understand human behavior, we may first need to understand the subconscious mind.

Dreams may be the expressions of the wants and desires of the subconscious mind. If dreams are understood, then we may be able to understand the true desires of the dreamer (e.g., the consumer). Dreams mainly occur in the rapid-eye movement (REM) stage of sleep, when brain activity is high and resembles that of being awake. During this time the pineal gland may secrete a neurotransmitter called Dimethyltryptamine (DMT). Some have proposed that the release of DMT is mechanism that causes the deep sleep visions.

The average individual spends eight hours a day sleeping. During this time, the individual may enter a dream world, where inhibitions can become freedoms. We spend a third of our lives sleeping, but we don't really understand dreaming. Why do we dream? Where does our conscious go for the vast majority of the night, every night?

Scholarly interest in the process and functions of dreaming has been present since Sigmund Freud's interpretations in the 1900s. The neurology of dreaming has remained misunderstood until recent developments. Modern techniques of brain imaging have provided new opportunities for the study of the subconscious mind. New technologies have provided a better understanding of dreams. However, many questions, in regards to their function and content, have yet to be answered.

In order to launch a successful product and conduct effective market research, organizations may need to understand the subconscious mind. Unfortunately, there is a fatal flaw in research of the dreaming, subconscious mind: the scope of the study. To understand dreams and the subconscious mind, research may need to be conducted on a global scale, not on an individual scale.

### BRIEF DESCRIPTION OF THE DRAWINGS

The accompanying drawings, which are incorporated in and constitute a part of this disclosure, illustrate various embodiments of the present disclosure. In the drawings:

FIG. 1A is a flow diagram illustrating a method for creating an animated representation of a single user's dream

FIG. 1B is a flow diagram illustrating a method for creating an animated representation of multiple dreams from multiple users;

FIG. 2 is a flow diagram illustrating a method for creating an illustrated representation of a user's dream;

FIG. 3 is a flow diagram illustrating a method for creating an interactive representation of multiple dreams from multiple users;

FIG. 4A is a flow diagram illustrating a method for creating a book representing a user's dream;

FIG. 4B is a flow diagram illustrating a method for creating a book representing multiple dreams from multiple users;

FIG. 5 is a flow diagram illustrating a method of creating a tangible representation of a user's dream;

FIG. 6 is a flow diagram illustrating a method of creating an immersive representation of multiple dreams from multiple users; and

FIG. 7 is a block diagram of a computing device consistent with embodiments of the present disclosure.

### DETAILED DESCRIPTION

The following detailed description refers to the accompanying drawings. Wherever possible, the same reference numbers are used in the drawings and the following description to refer to the same or similar elements. While many embodiments of the present disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. The present disclosure may contain headers. It should be understood that these headers are used as references and are not to be construed in any way as limiting upon the subjected matter disclosed under the header.

Overview

Embodiments of the present disclosure may provide a platform for dream sharing and visualization. First, a description of a dream may be received. The description may comprise a text and/or an image. Then the description may be published to the platform. A user may then pay a fee to have the dream visualized. In various embodiments, the description of the dream may be submitted for a vote by other users of the platform. When the dream is submitted to a vote and receives enough votes, the platform may provide a visual representation of the dream to the user who submitted the description of the dream. The visualization may then be published to the platform.

In accordance with to various embodiments of the present disclosure, the subconscious may be monetized through animated visual representations of dreams. The flexibility inherent within this methodology may allow for multiple embodiments to be created. In this way, users may help to create their own content. In addition, global dream studies may be conducted on these dreams to begin to understand the collective subconscious.

The advantage of this embodiment may be at least twofold: One, it may create products based on the subconscious mind of the consumer, circumventing the need for traditional market research. Two, it may affor an opportunity to broaden the scope of dreams studies, beyond the individual level, to the global collective level. Other advantages of one of more aspects will be apparent from a consideration of the drawings and ensuing description.

### Example Embodiments

Embodiments of the present disclosure relate to a platform for transforming subconscious dreams into visual or tangible representations of said dreams. The platform may be a system or method and it may, in some embodiments, be monetizable. Moreover, the dreams transformed by the platform may be documented for research purposes. Embodiments of the present disclosure may relate to a business methodology for monetizing the subconscious. Furthermore, embodiments of the present disclosure relate to a montetizable system to create tangible, immersive, and/or visual representations of users' dreams. Users of the platform will be hereinafter referred to as “Dreamers.” Throughout the present disclosure, “Dreamers” is intended to include all participants in the platform.

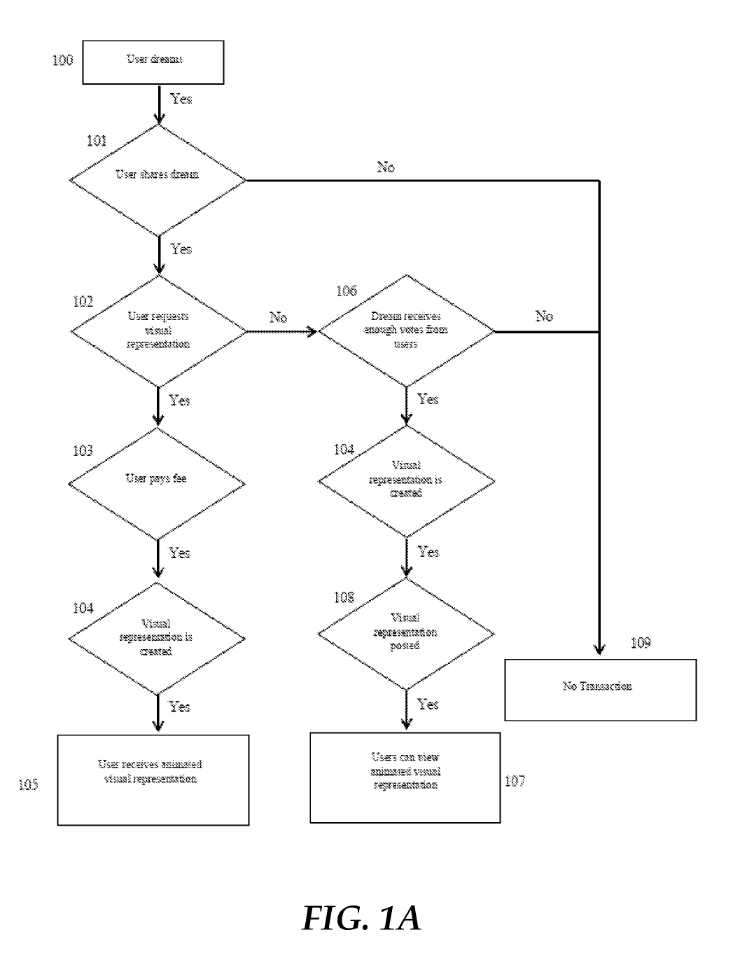

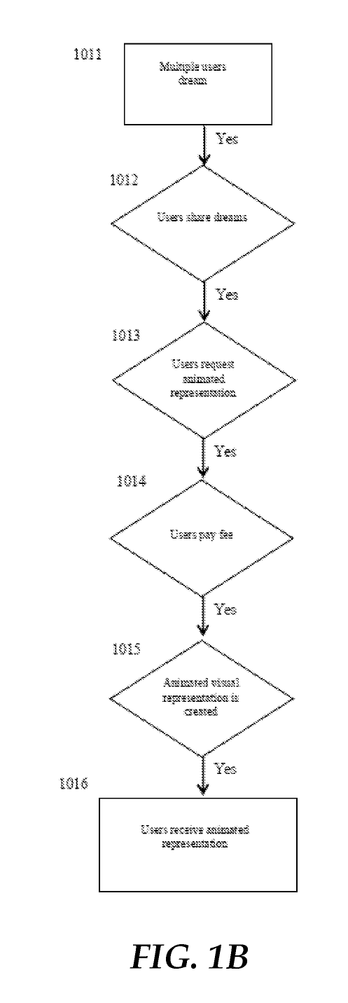

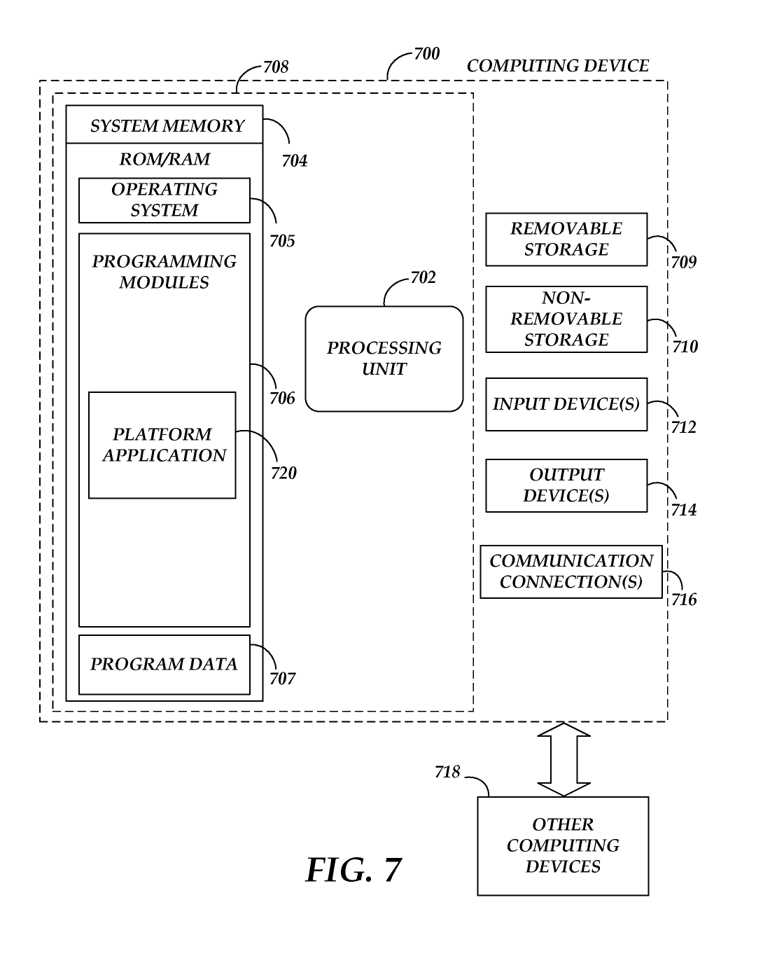

FIGS. 1-6 are flow charts setting forth the general stages involved in various methods consistent with various embodiments of the present disclosure. The methods may be implemented using a computing device 700 as described in more detail below with respect to FIG. 7. Moreover, various embodiments of the methods may be implemented in a system comprising a memory storage and a processing unit. Further still, various embodiments of the methods may be implemented in a cloud computing environment. Ways to implement the stages of the methods will be described in greater detail below.

FIG. 1A is a flow diagram illustrating one embodiment of transforming a Dreamer's dream into an animation. The method begins with the user dreaming 100. The Dreamer's dream visualization may be shared by the Dreamer on an online voting platform 101. If the Dreamer does not share the dream, then the process may stop and no transaction occurs 109. Once the Dreamer shares the dream, a request can be made for an animation of said dream 102. If the Dreamer requests an animation to be created to represent the dream, then a fee may need to be paid by the Dreamer 103. Once the fee is paid by the Dreamer, the visual representation of the dream in the form of an animation may be created by a team of animators 104. Once the animation is created, the Dreamer may receive the animation of said dream 105.

However, if the Dreamer does not request an animation, in some embodiments, a representation of the dream still has an opportunity to be animated. For example, the dream may be published and submitted to a voting process consistent with embodiments of the present disclosure. The dream must then receive enough votes by fellow Dreamers on the online voting platform 106. If the dream does not receive enough votes from Dreamers on the online voting platform, then the process may stop and no transaction will occur 109.

Dreamers on the online voting platform may be enabled to voice their preference in the form of voting. If enough Dreamers on the voting platform want the dream animated and the dream receives enough votes, then a visual representation of the dream may be created by a team of animators in the form of an animation 104. This animation may be shared on the online voting platform 108. Fellow Dreamers may be enabled to view this animation on the online voting platform 107.

Though the sharing and visualization of dreams on the platform, in various embodiments, the information may be collected from the dreams. Once enough dream information has been collected, we can begin to understand and analyze dreams on a collective scale. The understanding of the collective subconscious could provide insight into consumers' true desires that may currently be unavailable.

Current animations, such as cartoon shows, are created by media companies and animation studios. These shows are created based on traditional marketing research, which is ineffective because it elicits responses from the conscious mind. Various embodiments of the present disclosure may source the foundation of its animation from the subconscious mind of the Dreamers and allow them to create an animated representation of their own dreams. In various embodiments of the present disclosure, there may not be need for marketing research because the content and ideas are generated directly by the consumers and their subconscious mind. This may afford the Dreamers an opportunity to have a voice and presence in an animated show, thus creating a very interactive and immersive experience for said Dreamers.

To create an animation through various embodiments of the present disclosure, Dreamer must first have a dream 100. The dream may be a visual dream. The visual dream may be shared by the Dreamer through an online voting and collaboration platform 101 consistent with embodiments of the present disclosure. The dream 100 may be communicated to platform 101 through various media. For example, the dreamer may be enabled to upload pictures, texts, or videos that provide the details of what happened in their dreams. Possible prompts to help develop the animation from the viewers' dream may comprise: a) Describe what happened in your dream; b) Describe the surroundings in your dream; c) Describe the entities in your dream; and d) Include anything else you remember from your dream. In the future, this sharing process could change drastically as technology becomes more advanced.

Once the Dreamers share their dreams on said platform, they may have two options: They can request an animation of their dream or they can choose not to request an animation of their dreams 102. This request is very simple. Once the dream is shared, the Dreamers may have an option presented to them, asking them if they want to purchase a visual representation of their dreams. If yes, then they may pay the fees 103 and the animation may be created by an animation team 104.

Once the animation is created, Dreamers receive the animation that represents their respective dream 105. If the Dreamers select to not purchase a visual representation of their dreams, then the decision to animate the dream is moved to the Dreamer community on the online voting platform 106. If there are not enough Dreamers who vote to animate the dream, then no transaction occurs 109. If enough Dreamers vote yes to animate the dream, then the animation representing said dream is created by an animation team 104. This animation is posted on the online voting platform 108. Dreamers are able to view the animation of said dreams just by logging onto the online platform 107.

Additional embodiments are shown in FIGS. 1 B-6; in each case the embodiments for sharing and creating visualizations of dreams may produce various representations of the Dreamers' dreams. In FIG. 1 B, an animated representation of the shared Dreamers' dreams may be created. Multiple Dreamers dream 1011. They may all share their dreams on the online voting platform 1012. The Dreamers request an animated representation of their collective dreams 1013. If the dreams receive enough votes 1014, then an animated representation can be created 1015. Once the immersive representation has been created, then the Dreamers receive said representation 1016. Various embodiments may integrate many dreams from many Dreamers into an animated representation, thus forming an animated representation of the collective subconscious.

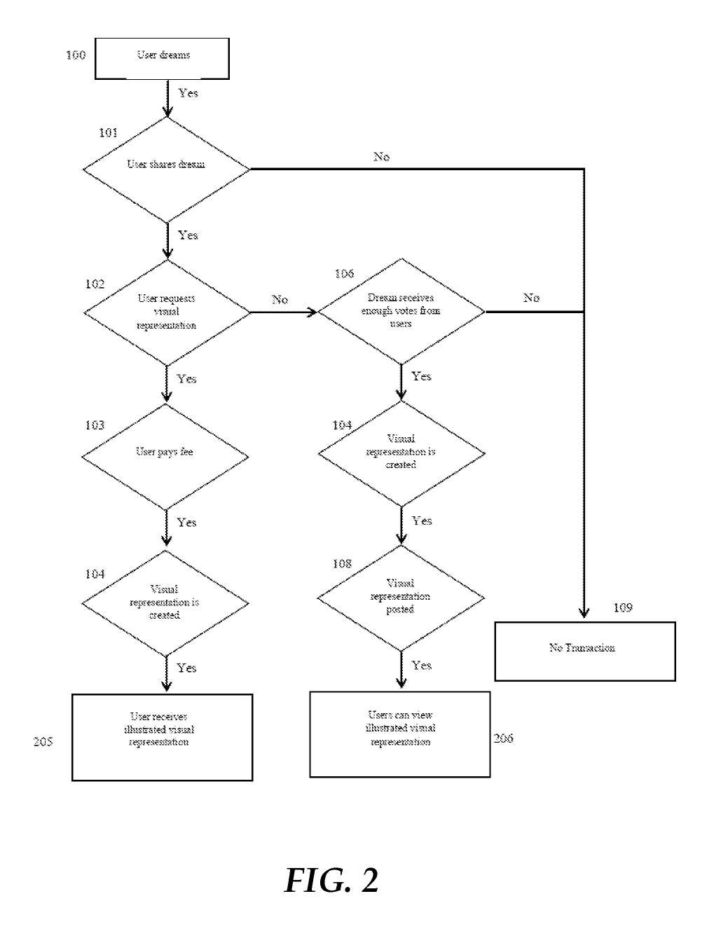

FIG. 2 is a flow diagram illustrating one embodiment of creating an illustrated representation of a user's dream. The method begins with the user dreaming 100. The Dreamer's dream visualization may be shared by the Dreamer on an online voting platform 101. If the Dreamer does not share the dream, then the process may stop and no transaction occurs 109. Once the Dreamer shares the dream, a request may be made for an illustration of said dream 102. If the Dreamer requests an illustration to be created to represent the dream, then a fee may be paid by the Dreamer 103. Once the fee is paid by the Dreamer, the visual representation of the dream in the form of an illustration may be created by an artist 104. Once the illustration is created, the Dreamer may receive the illustration of said dream 205. However, in some embodiments, if the Dreamer does not request an illustration, a representation of the dream still has an opportunity to be illustrated. As mentioned above, the dream may be submitted to a vote. The dream must receive enough votes by fellow Dreamers on the online voting platform 106. If the dream does not receive enough votes from Dreamers on the online voting platform, then the process may stop and no transaction will occur 109. If enough Dreamers on the voting platform want the dream illustrated and the dream receives enough votes, then a visual representation of the dream may created by an artist in the form of an illustration 104. This illustration may be shared on the online voting platform 108.

Fellow Dreamers may then view this illustration on the online voting platform 206. There are various possibilities that can embody an illustrated visual representation of the Dreamer's dream; these may comprise, but are not limited to: a) single illustrations, and b) a series illustrations that form a story, such as a comic strip or graphic novel. These representations are created by an artist, as opposed to a team of animators, as mentioned in the first embodiment.

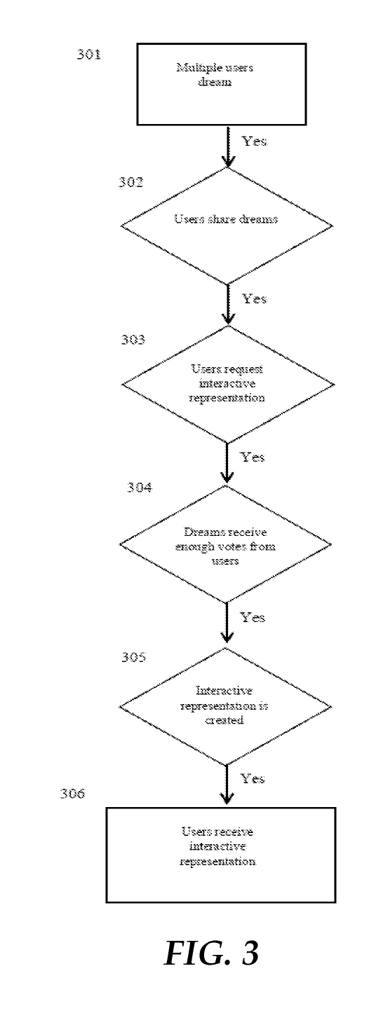

FIG. 3 is a flow diagram illustrating one embodiment of creating an interactive representation of multiple dreams from multiple users. Multiple Dreamers dream 301. They may all share their dreams on the online voting platform 302. The Dreamers may request an interactive representation of their collective dreams 303. If the dreams receive enough votes 304, then an interactive representation may be created 305. Once the interactive representation has been created, the Dreamers receive said representation 306. There are various possibilities that can embody an interactive visual representation of the Dreamer's dream; these may comprise, but are not limited to: a) video game and b) an interactive story board that allows individuals to manipulate characters and environments to create stories. This interactive visual representation may be created by a team of artists, designers, and programmers. Various embodiments of the disclosure may integrates many dreams from many Dreamers into an interactive representation, thus forming an interactive representation of the collective subconscious.

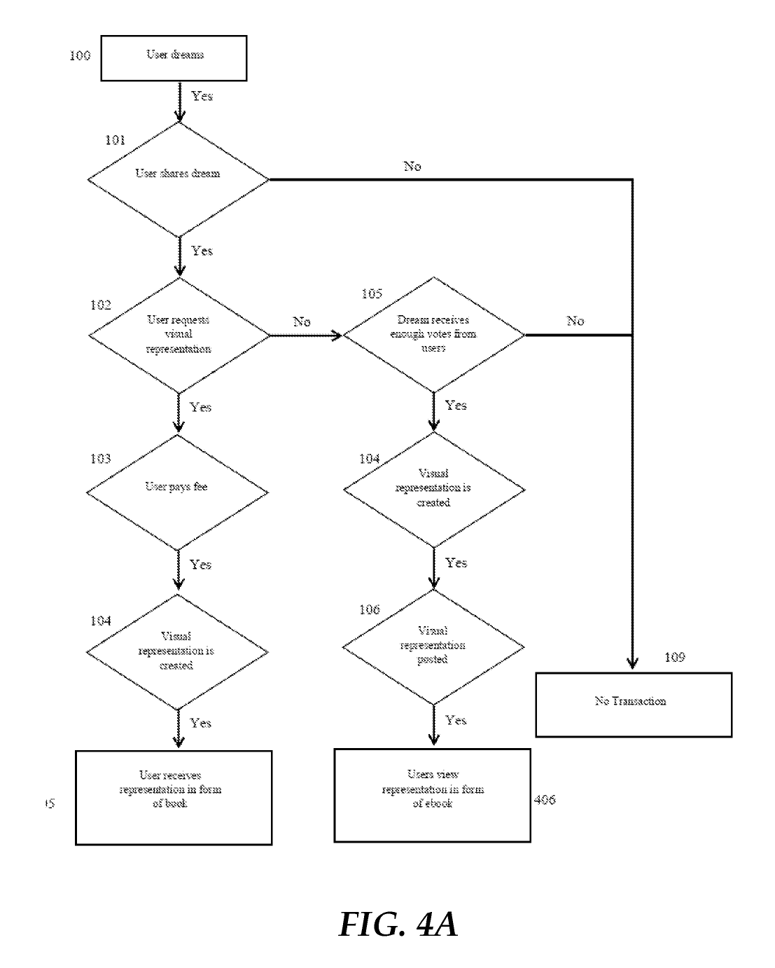

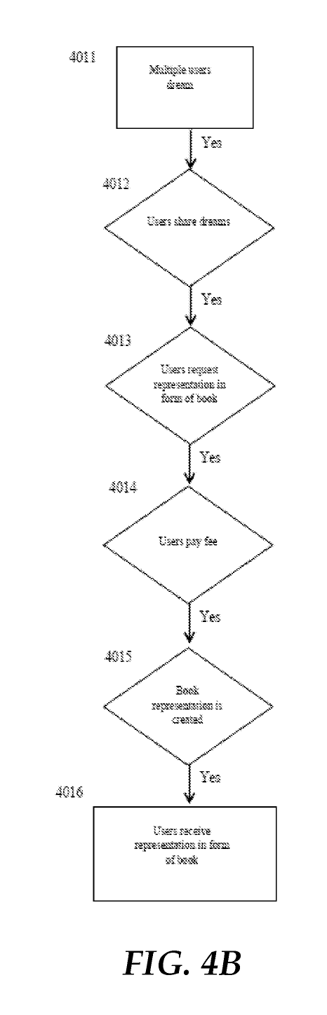

In FIG. 4A, a visual representation, in the form of a book, of the shared Dreamer's dream, may be created 405. This may be, for example, but not limited to an electronic book or a hardcopy book. The method begins with the Dreamer dreaming 100. The Dreamer's dream visualization is shared by the Dreamer on an online voting platform 101. If the Dreamer does not share the dream, then the process may stop and no transaction occurs 109. Once the Dreamer shares the dream, a request may be made for a book of said dream 102. If the Dreamer requests a book to be created to represent the dream, then a fee may be paid by the Dreamer 103. Once the fee is paid by the Dreamer, the visual representation of the dream in the form of a book may be created by a writer and/or artist 104. Once the book is created, the Dreamer may receive representation of said dream in the form of a book 405. However, if the Dreamer does not request a book, a representation of the dream still has an opportunity to be transformed into a book.

Again, the dream must receive enough votes by fellow Dreamers on the online voting platform 105. If the dream does not receive enough votes from Dreamers on the online voting platform, then the process may stop and no transaction will occur 109. If enough Dreamers on the voting platform want the dream created into a book, and the dream receives enough votes, then a representation of the dream may be created by a writer and/or artist in the form of an book 104. This illustration may be shared on the online voting platform 106. Fellow Dreamers can then view this book on the online voting platform 406. There are various possibilities that can embody a visual representation, in the form of a book, of the Dreamer's dream; these may comprise, but are not limited to: a) short story, b) novel, c) graphic novel and d) comic book. This representation may be created by artists and writers.

In FIG. 4B, a representation, in the form of a book, of the shared Dreamers' dreams may be created. Multiple Dreamers dream 4011. They all share their dreams on the online voting platform 4012. The Dreamers may request a book representation of their collective dreams 4013. If the dreams receive enough votes 4014, then a book representation can be created 4015. Once the representation has been created, then the Dreamers may receive said representation in the form of, for example, an electronic book 4016. This embodiment integrates many dreams from many Dreamers, thus forming representation of the collective subconscious in the form of a book.

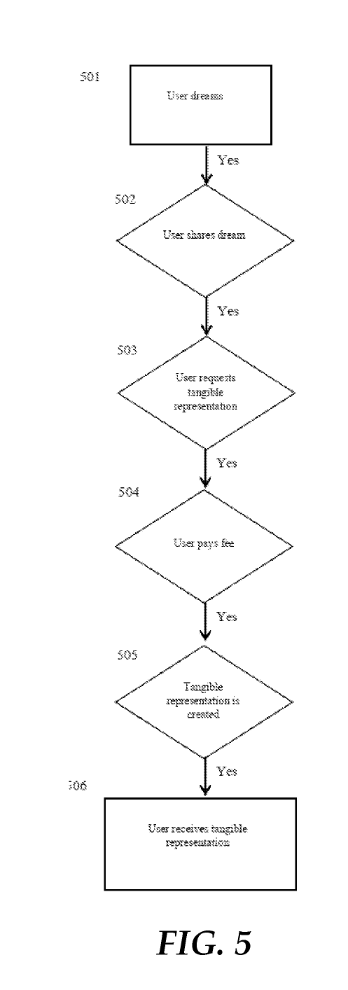

FIG. 5 is a flow diagram illustrating one embodiment of creating a tangible representation of a user's dream. The method begins with the Dreamer dreaming 501. The Dreamer's dream visualization may be shared by the Dreamer on an online voting platform 502. Once the Dreamer shares the dream, a request may be made for a tangible representation of said dream 503. If the Dreamer requests a tangible representation be created to represent the dream, then a fee may be paid by the Dreamer 504. Once the fee is paid by the Dreamer, the tangible representation of the dream may be created 505. Once the tangible representation is created, the Dreamer may receive said tangible representation 506. There are various possibilities that can embody tangible visual representation of the Dreamer's dream; these may comprise, but are not limited to: a) action figures, b) stuffed animals, c) construction toys, d) dolls, and e) board games. This interactive visual representation may be created by a team of artists and designers and manufactured to the specifications provided by the Dreamers.

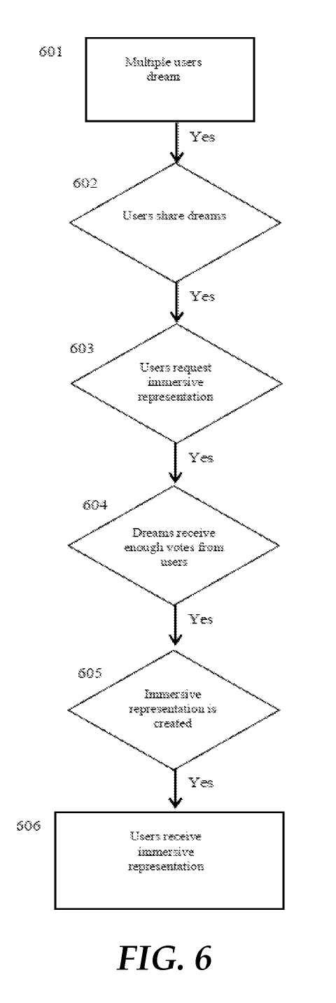

In FIG. 6, an immersive representation, of the shared Dreamers' dreams may be created. Multiple Dreamers dream 601. They may all share their dreams on the online voting platform 602. The Dreamers may request an immersive representation of their collective dreams 603. If the dreams receive enough votes 604, then the immersive representation may be created 605. Once the immersive representation has been created, then the users may receive and immerse themselves in said representation 606. Various embodiments may integrate many dreams from many Dreamers into an immersive representation, thus forming an immersive representation of the collective subconscious. There are various possibilities that can embody an immersive representation of the Dreamer's dreams; these may comprise, but are not limited to: a) adventure park and b) theme park. This representation may be created by a team of people from various industries.

By way of example and not limitation, a number of advantages of the various embodiments in the present disclosure may be:
a. Tapping into the infinite supply to dreams to afford Dreamers the opportunity for creative expression;
b. Allowing Dreamers to share and visualize dreams;
c. Allowing Dreamers to obtain visual representations of dreams;
d. Allowing Dreamers to create a representation of collective dreams;
e. Giving the dreamers creative license in creating an animation, book, game, etc, which fosters interaction with the consumer;
f. Circumventing the need for marketing research as it's method is founded directly in the subconscious;
g. Providing an opportunity to study the dream subconscious on a collective level rather than an individual level; and
h. Monetizing the subconscious by providing various possibilities of visualizations and representations of dreams.

Consistent with embodiments of the present disclosure, dreamers may share, discuss, and visualize their dreams through a platform, such as an online voting platform. There are many possibilities for creating representations of said dreams. Although the description above contains many specifications, these should not be construed as limiting the scope of the embodiments but merely providing illustrations of some of the several embodiments. For example, a movie based on the collective dreams of the Dreamers, could be created.

FIG. 7 is a block diagram for providing a computing device 700 consistent with embodiments of the present disclosure. In a basic configuration, computing device 700 may include at least one processing unit 702 and a system memory 704. Depending on the configuration and type of computing device, system memory 704 may comprise, but is not limited to, volatile (e.g. random access memory (RAM)), non-volatile (e.g. read-only memory (ROM)), flash memory, or any combination. System memory 704 may include operating system 705, one or more programming modules 706, and may include a program data 707. Operating system 705, for example, may be suitable for controlling computing device 700′s operation. In one embodiment, programming modules 706 may include, for example, a smart watch application 720. Other applications may be operative with embodiments of the disclosure, such as, but not limited to, for example, smart phone applications or personal digital assistant (PDA) applications. Furthermore, embodiments of the present disclosure may be practiced in conjunction with a graphics library, other operating systems, or any other application program and is not limited to any particular application or system. This basic configuration is illustrated in FIG. 7 by those components within a dashed line 708.

Computing device 700 may have additional features or functionality. For example, computing device 700 may also include additional data storage devices (removable and/or non-removable) such as, for example, magnetic disks, optical disks, or tape. Such additional storage is illustrated in FIG. 7 by a removable storage 709 and a non-removable storage 710. Computer storage media may include volatile and nonvolatile, removable and non-removable media implemented in any method or technology for storage of information, such as computer readable instructions, data structures, program modules, or other data. System memory 704, removable storage 709, and non-removable storage 710 are all computer storage media examples (i.e memory storage.) Computer storage media may include, but is not limited to, RAM, ROM, electrically erasable read-only memory (EEPROM), flash memory or other memory technology, optical storage, magnetic disk storage or other magnetic storage devices, or any other medium which can be used to store information and which can be accessed by computing device 700. Any such computer storage media may be part of device 700. Computing device 700 may also have input device(s) 712 such as a keyboard, a mouse, a pen, a sound input device, a touch input device, etc. Output device(s) 714 such as a display, speakers, a printer, etc. may also be included. The aforementioned devices are examples and others may be used.

Computing device 700 may also contain a communication connection 716 that may allow device 700 to communicate with other computing devices 718, such as over a network in a distributed computing environment, for example, an intranet or the Internet. Communication connection 716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 704, including operating system 705. While executing on processing unit 702, programming modules 706 (e.g. dream sharing, voting, and/or visualization application) may perform processes including, for example, one or more methods as described above. The aforementioned process is an example, and processing unit 702 may perform other processes. Other programming modules that may be used in accordance with embodiments of the present disclosure may include electronic mail and contacts applications, word processing applications, spreadsheet applications, database applications, slide presentation applications, drawing or computer-aided application programs, etc.

Generally, consistent with embodiments of the present disclosure, program modules may include routines, programs, components, data structures, and other types of structures that may perform particular tasks or that may implement particular abstract data types. Moreover, embodiments of the present disclosure may be practiced with other computer system configurations, including hand-held devices, multiprocessor systems, microprocessor-based or programmable consumer electronics, minicomputers, mainframe computers, and the like. Embodiments of the present disclosure may also be practiced in distributed computing environments where tasks are performed by remote processing devices that are linked through a communications network. In a distributed computing environment, program modules may be located in both local and remote memory storage devices.

Furthermore, embodiments of the present disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the present disclosure may also be practiced using other technologies capable of performing logical operations such as, for example, AND, OR, and NOT, including but not limited to mechanical, optical, fluidic, and quantum technologies. In addition, embodiments of the present disclosure may be practiced within a general purpose computer or in any other circuits or systems.

Embodiments of the present disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random access memory (RAM), a read-only memory (ROM), an erasable programmable read-only memory (EPROM or Flash memory), and an optical fiber. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

While the specification includes examples, the present disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the present disclosure.

The following is claimed:

1. A method comprising:
a. Receiving at least one input from at least one user, the at least one input being associated with content relating to a dream of the at least one user;
b. Receiving a request for a visual representation of the at least one input;
c. Creating the visual representation; and
d. Providing the at least one user with the visual representation.

2. The method of claim 1, further comprising: publishing the at least one input to a platform.

3. The method of claim 2, further comprising: submitting the at least one input to a vote.

4. The method of claim 3, further comprising: receiving, through the platform, votes in favor of a creation of a visual representation of the at least one input.

5. The method of claim 4, further comprising: determining if the votes reach a predetermined threshold.

6. The method of claim 5, when the votes reach the predetermined threshold, further comprising:
a. Creating the visual representation; and
b. Publishing the visual representation.

7. A computer-readable medium comprising a set of instruction which, when executed by a computer, perform a method comprising:
a. Receiving a description of a dream of a user;
b. Publishing the description of the dream to an online platform;
c. Enabling users of the platform to vote in favor of the dream;
d. Determining if votes for the dream have reached a threshold; and
e. When the votes for the dream have reached the threshold:
i. Creating the visual representation of the dream, and
ii. Providing the at least one user with the visual representation.

8. The computer-readable medium of claim 7, further comprising: receiving a payment for the creation of the visual representation.

9. The computer-readable medium of claim 7, wherein the visual representation comprises at least one of the following: an illustration, an animation, an interactive interface, a book, a multimedia representation, a video game, a tangible object, and an immersive.

10. The computer-readable medium of claim 7, wherein the description of the dream comprises at least one of the following: a text and an image.

11. A system comprising:
a. A memory storage; and
b. A processing unit coupled to the memory storage; the processing unit being configured to:
i. Receive a description of a dream of a user;
ii. Publish the description of the dream to an online platform;
iii. Enable users of the platform to vote in favor of the dream;
iv. Determine if votes for the dream have reached a threshold; and
v. When the votes for the dream have reached the threshold:
1. Create the visual representation of the dream; and
